(19) United States
(12) Patent Application Publication
(10) Pub. No.: US 2017/0364655 A1
(43) Pub. Date: Dec. 21, 2017

(54) MONITORING ADHERENCE TO A HEALTHCARE PLAN

(71) Applicant: Aftechmobile Inc. (d/b/a Mobrise Inc.), Sterling, VA (US)

(72) Inventor: Arshad Farooqi, Potomac Falls, VA (US)

(21) Appl. No.: 15/624,037

(22) Filed: Jun. 15, 2017

Related U.S. Application Data
(60) Provisional application No. 62/350,205, filed on Jun. 15, 2016.

(51) Int. Cl. G06F 19/00

(52) U.S. Cl. CPC G06F 19/3418; G06F 19/3456; G06Q 2220/00; G06F 19/3487

(57) ABSTRACT

Various aspects and embodiments relate to a healthcare adherence monitoring system (HAMS) that can be used to monitor adherence of a healthcare recipient to a healthcare plan (e.g., a medication, exercise, and nutrition regimen). The HAMS may receive a personalized prescription including one or more pre-coded prescription components and/or customized recommendations of the healthcare plan from a healthcare provider device via a communication network. The HAMS may then incorporate the received personalized prescription for the healthcare recipient into a healthcare mobile application that a healthcare recipient device can access via the communication network. The HAMS may further dynamically update the personalized prescription based on configuration parameters received from the healthcare mobile application on the healthcare recipient device via the communication network and communicate with the healthcare mobile application on the healthcare recipient device via the communication network to monitor the healthcare recipient's adherence to the dynamically updated personalized prescription.

### CROSS-REFERENCE TO RELATED APPLICATIONS

The present application claims priority to U.S. Provisional Patent Application Ser. No. 62/350,205, entitled “Monitoring Adherence To A Healthcare Plan,” filed Jun. 15, 2016, which is hereby incorporated by reference in its entirety.

### TECHNICAL FIELD

Various aspects and embodiments generally relate to healthcare, and more particularly, to monitoring adherence of a healthcare recipient to a healthcare plan.

### BACKGROUND

Consumers and business entities are increasingly using mobile devices such as smart phones, tablet computing devices, personal digital assistants, other handheld devices, etc., to perform various personal, work, and business or enterprise related tasks. In addition to using mobile devices for different forms of communication, for example, voice communication, messaging, etc., these mobile devices also allow consumers and business entities to perform other functions such as accessing and displaying websites, sending and receiving electronic mails, capturing and displaying images, playing videos, music and other forms of audio, social networking, etc. These functions are typically performed by software applications that are either built into the mobile devices or that are run on top of operating systems of the mobile devices.

Competing mobile device manufacturers, developers, and other service providers typically offer hardware and software in the form of mobile operating systems or in some cases, a combination of both hardware and software, which has led to the development of dissimilar and incompatible mobile device platforms and environments due to the use of different operating systems which have their own programming environments and software development tools. Developing mobile applications for these different operating systems and mobile devices requires varying design techniques, programming languages, separate operating system specific or device specific software code and compilations, etc. Therefore, developers find it difficult and challenging to develop mobile applications that are portable across multiple types of mobile devices. Furthermore, developers require specific technical knowledge and expertise in software programming with specific programming languages and an understanding of specific mobile operating system platforms for developing mobile applications for different types of mobile operating systems and/or mobile devices. Since a high level of skill and expertise is needed to create, deploy and update mobile applications, many non-technical users and users who lack experience in a specific programming language or a specific mobile operating system cannot develop and/or distribute mobile applications.

Conventional mobile development platforms typically allow users to develop mobile applications of a particular type that cannot be configured or changed and therefore limit the development and utilization of various features and specific functions included in mobile devices and their respective mobile operating systems. The scope of these mobile applications are limited to consumer applications such as access to website content, graphics, video, books, links to a social profile, etc. These mobile development platforms allow non-technical users to develop only generic business-to-consumer (B2C) mobile applications with simplified rules. B2C mobile applications comprising metadata (e.g., an application type) are relatively easier to develop and are supported by most mobile development platforms. There is therefore a need for providing methods and systems that can unify similar or disparate software components from multiple data sources and enable non-technical users to create mobile applications that suit different needs without requiring coding or other technical expertise.

Moreover, conventional mobile development platforms only provide access to a single layer of data that allow, for example, web pages to be assembled on the fly but do not provide support for hierarchical layers of data, data interaction, and business rules that allow non-technical enterprise users to quickly assemble software components to develop enhanced enterprise grade mobile applications. Moreover, conventional mobile development platforms do not allow backend integration of the developed mobile application with the user's computing device or with backend databases for non-technical users.

Furthermore, context and behavioral elements, for example, user behavior, user intent, etc., varies from one user to another, and typical mobile application development platforms do not provide components or tools that are adaptable based on the context and behavioral elements. Moreover, conventional mobile application development platforms do not support dynamic data mapping for establishing a link between the data being rendered and multiple data sources prior to creation of a mobile application. Furthermore, non-technical users may not be aware of enhanced functions and features that can be implemented in a mobile application for enhancing the mobile application.

Wearable devices and associated technology are becoming more widespread and are being used in everyday life for carrying out various activities. Moreover, due to recent adoption of policies, for example, bring your own devices (BYOD), organizations are in constant need of mobile application development platforms that can be used by non-technical users to design and develop complex mobile applications that can be deployed across various devices including wearable devices with varying degrees of form factors, to securely manage upgrades and downloads through an application store, and to manage usage statistics to gain better visibility on mobile application usage. Typical mobile application development platforms do not allow non-technical users to develop and deploy complex business-to-business (B2B) mobile applications for these wearable devices.

Furthermore, machine-to-machine (M2M) applications use a device, for example, a sensor, a meter, etc., to capture an event, for example, temperature, an inventory level, etc., relay the captured event, for example, through a wireless, wired or hybrid network to a mobile application, and translate the captured event into meaningful information. The M2M applications allow both wireless and wired systems to communicate with other devices of the same ability. Typical mobile application development platforms do not combine device data, for example, sensor data with application business processes to enable decision making based on real time analytics and mashup applications.

Hence, there is a long felt but unresolved need for a computer implemented method and system that provides encapsulated pre-coded software components to perform multiple functions in the form of a software package and enterprise grade mobile applications that are connected with backend databases, enables creation of enhanced software components or widgets without coding, provides support for native, web and cross platform mobile applications, provides an interface to dynamically map backend data sources and add new fields on the fly, provides connection of data from multiple data sources, enables creation of complex business-to-business (B2B) mobile applications that require complex business rules and data validation, allows development of M2M applications or sensor specific applications that combine sensor data with business processes and creation of process and sensor composite or mashup applications, and implements application programming interfaces for backend integration of the developed mobile application with the user's computing device and with backend databases. Furthermore, there is a need for a computer implemented method and system that enables a user to create mobile applications with software components that comprise hierarchical layers of data and that adapt based on the context and behavioral elements of the user when deployed on mobile devices. Furthermore, there is a need for a computer implemented method and system that enables non-technical users to develop and deploy complex mobile applications for various devices including the wearable devices without any programming.

### SUMMARY

The following presents a simplified summary relating to one or more aspects and/or embodiments disclosed herein. As such, the following summary should not be considered an extensive overview relating to all contemplated aspects and/or embodiments, nor should the following summary be regarded to identify key or critical elements relating to all contemplated aspects and/or embodiments or to delineate the scope associated with any particular aspect and/or embodiment. Accordingly, the following summary has the sole purpose to present certain concepts relating to one or more aspects and/or embodiments relating to the mechanisms disclosed herein in a simplified form to precede the detailed description presented below.

According to various aspects, as will be described in further detail herein, a healthcare adherence monitoring system (HAMS) may comprise at least one processor configured to execute computer program instructions for monitoring adherence or compliance of a healthcare recipient to a healthcare plan. In an embodiment, the HAMS provides multiple pre-coded prescription components to allow a healthcare provider to select one or more of the pre-coded prescription components constituting a personalized prescription of the healthcare plan for the healthcare recipient. The pre-coded prescription components are executable by one or more processors of the HAMS and are adaptable based on context and behavioral elements. The pre-coded prescription components comprise hierarchical layers of data, interactive elements that enable interactions with the data, and predetermined criteria.

According to various aspects, as will be described in further detail herein, the HAMS may receive a personalized prescription comprising one or more of the pre-coded prescription components and/or customized recommendations of the healthcare plan from a healthcare provider device via a communication network. The HAMS incorporates the received personalized prescription for the healthcare recipient in a healthcare mobile application accessible by a healthcare recipient device via the communication network. The HAMS dynamically updates the personalized prescription based on configuration parameters received from the healthcare mobile application on the healthcare recipient device via the communication network. The HAMS monitors adherence of the healthcare recipient to the dynamically updated personalized prescription in communication with the healthcare mobile application on the healthcare recipient device via the communication network.

According to various aspects, a method for monitoring adherence of a healthcare recipient to a healthcare plan may comprise receiving, at a healthcare adherence monitoring system, a personalized prescription that comprises one or more healthcare recommendations received from a healthcare provider device via a communication network, incorporating, at the healthcare adherence monitoring system, the personalized prescription into a healthcare mobile application, sending, by the healthcare adherence monitoring system, the healthcare mobile application incorporating the personalized prescription to a healthcare recipient device via the communication network, and monitoring, at the healthcare adherence monitoring system, adherence of the healthcare recipient to the personalized prescription based on adherence data received from the healthcare mobile application on the healthcare recipient device via the communication network.

According to various aspects, the method for monitoring the adherence of the healthcare recipient to the healthcare plan may further comprise providing, by the healthcare adherence monitoring system, an interface that enables the healthcare provider device to drag and drop one or more pre-coded prescription components to create the healthcare mobile application, wherein the pre-coded prescription components include executable computer program code encapsulated to perform predefined functions corresponding to the personalized prescription.

According to various aspects, the method for monitoring the adherence of the healthcare recipient to the healthcare plan may further comprise dynamically updating the personalized prescription based on one or more configuration parameters received from the healthcare mobile application on the healthcare recipient device via the communication network, dynamically generating an adherence report based on the monitored adherence of the healthcare recipient to the dynamically updated personalized prescription, and transmitting the adherence report to the healthcare recipient device and/or the healthcare provider device via the communication network.

According to various aspects, the method for monitoring the adherence of the healthcare recipient to the healthcare plan may further comprise storing, at the healthcare adherence monitoring system, data related to the adherence of the healthcare recipient to the personalized prescription, wherein the data related to the adherence of the healthcare recipient to the personalized prescription is accessible to the healthcare provider device via the communication network and receiving, at the healthcare adherence monitoring system, one or more customized interventions from the healthcare provider device via the communication network, the one or more customized interventions including one or more steps to be taken by the healthcare recipient for a healthier lifestyle. As such, in various embodiments, the one or more customized interventions may be sent to the healthcare recipient device via the communication network. Alternatively and/or additionally, the stored data related to the adherence of the healthcare recipient to the personalized prescription may be integrated with one or more medical records of the healthcare recipient, which may be used in combination to predict a likelihood of one or more health outcomes for the healthcare recipient based on the stored data related to the adherence of the healthcare recipient to the personalized prescription in combination with the one or more medical records of the healthcare recipient.

According to various aspects, the method for monitoring the adherence of the healthcare recipient to the healthcare plan may further comprise receiving, at the healthcare adherence monitoring system, information from the healthcare recipient device inviting one or more caregivers to track the adherence of the healthcare recipient to the personalized prescription and sending, by the healthcare adherence monitoring system, the healthcare mobile application incorporating the personalized prescription to one or more devices associated with the one or more caregivers via the communication network. As such, in various embodiments, the healthcare adherence monitoring system may monitor adherence of the healthcare recipient to the personalized prescription based on adherence data received from the healthcare mobile application on the one or more devices associated with the one or more caregivers.

According to various aspects, the method for monitoring the adherence of the healthcare recipient to the healthcare plan may further comprise storing, at the healthcare adherence monitoring system, the adherence data received from the healthcare mobile application on the healthcare recipient device as one or more blocks in an adherence blockchain, wherein each of the one or more blocks in the adherence blockchain includes a cryptographic hash of the adherence data stored in the respective block. As such, in various embodiments, the adherence data stored as the one or more blocks in the adherence blockchain may be converted into healthcoins, wherein the healthcoins may be accessible to one or more entities via the communication network to prescribe or regulate one or more of healthcare premiums or incentives for adhering to the personalized prescription.

According to various aspects, a healthcare adherence monitoring system may comprise a network interface configured to communicate via a communication network and one or more processors configured to receive, via the network interface, a personalized prescription comprising one or more healthcare recommendations received from a healthcare provider device, incorporate the personalized prescription into a healthcare mobile application, send, via the network interface, the healthcare mobile application incorporating the personalized prescription to a healthcare recipient device, and monitor adherence of the healthcare recipient to the personalized prescription based on adherence data received from the healthcare mobile application on the healthcare recipient device via the communication network.

According to various aspects, a non-transitory computer-readable storage may comprise computer-executable code configured to cause at least one processor to receive a personalized prescription, wherein the personalized prescription comprises one or more healthcare recommendations received from a healthcare provider device via a communication network, incorporate the personalized prescription into a healthcare mobile application, send the healthcare mobile application incorporating the personalized prescription to a healthcare recipient device via the communication network, and monitor adherence of the healthcare recipient to the personalized prescription based on adherence data received from the healthcare mobile application on the healthcare recipient device via the communication network.

According to various aspects, another method for monitoring adherence of a healthcare recipient to a healthcare plan may comprise receiving a personalized prescription at a healthcare adherence monitoring system, wherein the personalized prescription received at the healthcare adherence monitoring system comprises one or more healthcare recommendations received from a healthcare provider device via a communication network, incorporating, at the healthcare adherence monitoring system, the personalized prescription into a healthcare mobile application, sending, by the healthcare adherence monitoring system, the healthcare mobile application incorporating the personalized prescription to a healthcare recipient device via the communication network, and storing, at the healthcare adherence monitoring system, adherence data received from the healthcare mobile application on the healthcare recipient device via the communication network as one or more blocks in an adherence blockchain, wherein each of the one or more blocks in the adherence blockchain includes a cryptographic hash of the adherence data stored in the respective block. As such, in various embodiments, the adherence data stored as the one or more blocks in the adherence blockchain may be converted into healthcoins, which may be accessible to one or more entities via the communication network to prescribe or regulate one or more of healthcare premiums or incentives for adhering to the personalized prescription.

Other objects and advantages associated with the aspects and embodiments disclosed herein will be apparent to those skilled in the art based on the accompanying drawings and detailed description.

### BRIEF DESCRIPTION OF THE DRAWINGS

A more complete appreciation of the various aspects and embodiments described herein and many attendant advantages thereof will be readily obtained as the same becomes better understood by reference to the following detailed description when considered in connection with the accompanying drawings which are presented solely for illustration and not limitation, and in which:

FIG. 1 illustrates an exemplary for monitoring adherence of a healthcare recipient to a healthcare plan, according to various aspects.

FIG. 2 illustrates an exemplary communication between a healthcare adherence monitoring system, a data source, patient applications, and an adherence data warehouse, according to various aspects.

FIG. 3 illustrates exemplary healthcare entities connected to a communication network of the healthcare adherence monitoring system, according to various aspects.

FIG. 4 illustrates an exemplary high-level architecture of a computer implemented system for creating a mobile application for a user device, according to various aspects.

FIG. 5 illustrates an exemplary computer implemented system for creating a mobile application for a user device, showing different modules of a mobile application development software, according to various aspects.

FIG. 6 illustrates an exemplary computer system architecture employed by the mobile application development software for creating a mobile application for a user device, according to various aspects.

### DETAILED DESCRIPTION

Various aspects and embodiments are disclosed in the following description and related drawings to show specific examples relating to exemplary aspects and embodiments. Alternate aspects and embodiments will be apparent to those skilled in the pertinent art upon reading this disclosure, and may be constructed and practiced without departing from the scope or spirit of the disclosure. Additionally, well-known elements will not be described in detail or may be omitted so as to not obscure the relevant details of the aspects and embodiments disclosed herein.

The word “exemplary” is used herein to mean “serving as an example, instance, or illustration.” Any embodiment described herein as “exemplary” is not necessarily to be construed as preferred or advantageous over other embodiments. Likewise, the term “embodiments” does not require that all embodiments include the discussed feature, advantage, or mode of operation.

The terminology used herein describes particular embodiments only and should not be construed to limit any embodiments disclosed herein. As used herein, the singular forms “a,” “an,” and “the” are intended to include the plural forms as well, unless the context clearly indicates otherwise. Those skilled in the art will further understand that the terms “comprises,” “comprising,” “includes,” and/or “including,” as used herein, specify the presence of stated features, integers, steps, operations, elements, and/or components, but do not preclude the presence or addition of one or more other features, integers, steps, operations, elements, components, and/or groups thereof.

Further, various aspects and/or embodiments may be described in terms of sequences of actions to be performed by, for example, elements of a computing device. Those skilled in the art will recognize that various actions described herein can be performed by specific circuits (e.g., an application specific integrated circuit (ASIC)), by program instructions being executed by one or more processors, or by a combination of both. Additionally, these sequences of actions described herein can be considered to be embodied entirely within any form of non-transitory computer-readable medium having stored thereon a corresponding set of computer instructions that upon execution would cause an associated processor to perform the functionality described herein. Thus, the various aspects described herein may be embodied in a number of different forms, all of which have been contemplated to be within the scope of the claimed subject matter. In addition, for each of the aspects described herein, the corresponding form of any such aspects may be described herein as, for example, “logic configured to” and/or other structural components configured to perform the described action.

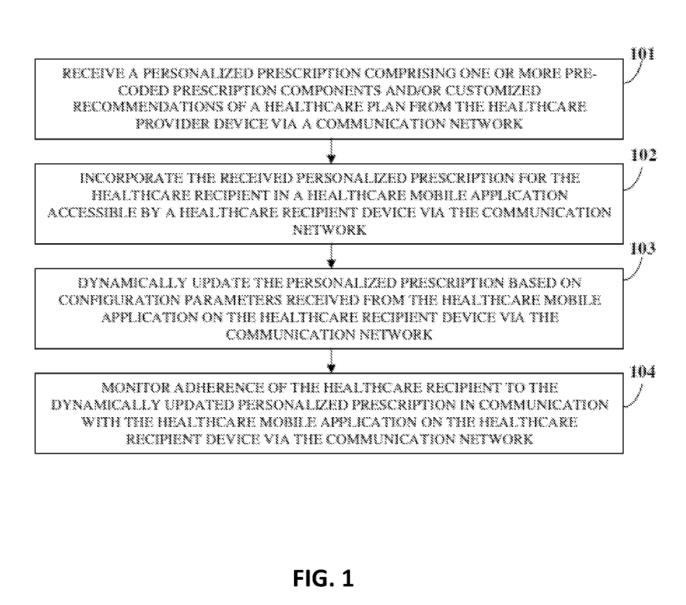

According to various aspects, FIG. 1 illustrates an exemplary method for monitoring adherence of a healthcare recipient to a healthcare plan. As used herein, the term “healthcare recipient” may generally refer to a person or an entity, for example, a patient who receives healthcare services from a healthcare provider. Also, as used herein, the term “healthcare provider” may generally refer to a person or an entity, for example, a medical practitioner, a medical specialist, a health specialist, a physician, a doctor, a dentist, a surgeon, a nurse, a therapist, a nutritionist, a pharmacist, a clinical trial professional, a clinical study professional, a health insurance company, a health maintenance organization, a caregiver, etc., that provides healthcare services, for example, medical treatment, medication, dental treatment, health insurance, etc., to a healthcare recipient. In various embodiments, the method shown in FIG. 1 may be implemented in a healthcare adherence monitoring system (HAMS) comprising at least one processor configured to execute computer program instructions for monitoring adherence of a healthcare recipient to a healthcare plan.

For example, in various embodiments, the HAMS may provide multiple pre-coded prescription components corresponding to a prescription of a healthcare plan, wherein the pre-coded prescription components may include, without limitation, smart widgets, micro widgets, etc. As used herein, “pre-coded prescription components” refers to pieces of pre-written computer program code encapsulated to perform predefined functions corresponding to a prescription of a healthcare plan, in the form of a software package or a software model, for example, a smart widget, a micro widget, etc. The HAMS allows a healthcare provider using a healthcare provider device to select one or more of the pre-coded prescription components constituting a personalized prescription of the healthcare plan for the healthcare recipient. As used herein, “healthcare provider device” refers to a user device, for example, a mobile phone, a smartphone, a tablet computing device, a personal computer, a laptop, a wearable computing device such as the Google Glass® of Google Inc., the Apple Watch® of Apple Inc., etc., capable of rendering the pre-coded prescription components for selection by the healthcare provider and communicating with the HAMS over a communication network, for example, the Internet, a wireless network, a mobile telecommunication network, etc. The personalized prescription defines, for example, exercise, nutrition, and medication recommended for the healthcare recipient by the healthcare provider.

The pre-coded prescription components are executable by one or more processors of the healthcare adherence monitoring system (HAMS) and are adaptable based on context and behavioral elements, for example, preferences of a user, habits of a user, etc. The pre-coded prescription components such as smart widgets contain elements that are adaptable to the behavioral mindset of a mobile native user while the user accesses the smart widgets from a user device. For example, a healthcare mobile application using the pre-coded prescription components on the healthcare provider device can highlight a list of healthcare recipients to follow up with based on contextual and behavioral elements such as a location and healthcare requirements of the healthcare recipient. The pre-coded prescription components further comprise hierarchical layers of data, for example, multiple layers of pages, interactive elements that enable interactions with the data, and predetermined criteria. As used herein, “interactive element” refers to any interface component configured to obtain data or feedback from a user, for example, a healthcare recipient or a healthcare provider, transform the obtained data into one or more visual entities, enable real-time user interactions within a graphical user interface of a user device, for example, a healthcare recipient device, the healthcare provider device, etc. The predetermined criteria comprise, for example, norms and protocols to be followed in healthcare. As an example, a norm followed in healthcare defines that a healthcare recipient needs to undergo a general health checkup before consultation with a healthcare provider. In an embodiment, the HAMS represents the pre-coded prescription components as reusable graphical user interface elements on the healthcare provider device. In another embodiment, the pre-coded prescription components, for example, smart widgets are preassembled based on the healthcare plan, healthcare process flows, and healthcare requirements of the healthcare recipient.

Referring specifically to FIG. 1, at block 101, the HAMS may receive a personalized prescription comprising one or more pre-coded prescription components selected by the healthcare provider from the healthcare provider device and/or customized recommendations of the healthcare plan from the healthcare provider device via a communication network, for example, the Internet, a wireless network, a mobile telecommunication network, etc. As used herein, “customized recommendations” refer to prescription guidelines customized for the healthcare recipient by the healthcare provider. The customized recommendations comprise, for example, types of exercises suitable for the healthcare recipient, duration of the exercises, dose of medication, guidelines for nutrition, start dates of the exercises and medication, etc. For example, in various embodiments, the HAMS may generate the personalized prescription (e.g., based on the patient's laboratory data and/or other suitable data), wherein the personalized prescription may include medication, exercise, and nutrition regimens. The HAMS-generated personalized prescription may then be presented to the healthcare provider (e.g., while the healthcare provider is seeing the patient/healthcare recipient). The healthcare provider can further customize the personalized prescription and send personalized prescriptions to be displayed on a mobile application for the healthcare recipient, which may be created in the manner described in further detail below. Alternatively, the healthcare provider can create and send one or more parameters for the healthcare mobile application that incorporates the personalized prescription to the HAMS. As such, the various aspects and embodiments described herein contemplate that the personalized prescription received from the healthcare provider at block 101 may include one or more customizations to a personalized prescription initially generated at the HAMS and/or one or more parameters that the healthcare provider creates and sends to the HAMS without any initial input from the HAMS.

According to various aspects, at block 102, the HAMS may incorporate the received personalized prescription for the healthcare recipient in a healthcare mobile application accessible by a healthcare recipient device via the communication network. As used herein, “healthcare recipient device” refers to a user device, for example, a mobile phone, a smartphone, a tablet computing device, a personal computer, a laptop, a wearable computing device such as the Google Glass® of Google Inc., the Apple Watch® of Apple Inc., etc., capable of rendering and invoking the healthcare mobile application and communicating with the HAMS over the communication network. In an embodiment, the HAMS creates the healthcare mobile application based on the received personalized prescription comprising the selected pre-coded prescription components for the healthcare recipient. The healthcare provider, for example, a physician, at a point of care, provides a customized set of recommendations, for example, for medication, exercise, and nutrition for healthcare recipients in the form of personalized prescriptions comprising, in an embodiment, the selected pre-coded prescription components to the HAMS through the communication network for the creation of the healthcare mobile application, which then can be accessed by the healthcare recipients through the communication network via the healthcare mobile application on their healthcare recipient devices. The healthcare provider can also drag and drop the selected pre-coded prescription components into the HAMS that then creates customized healthcare mobile applications for the healthcare recipients.

In an embodiment, the HAMS may send the received personalized prescription comprising the customized recommendations from the healthcare provider to a preexisting healthcare mobile application deployed on the healthcare recipient device via the communication network. The healthcare mobile application is either already resident on the healthcare recipient device or is downloaded onto the healthcare recipient device from a network location. For example, healthcare recipients who are first time users of the HAMS can download the healthcare application onto their user devices from an application store on the Internet. In this embodiment, the HAMS sends the customized recommendations to be dynamically updated on the preexisting healthcare mobile application deployed on the healthcare recipient device via the communication network.

In various embodiments, at block 103, the HAMS may dynamically update the personalized prescription based on configuration parameters received from the healthcare mobile application on the healthcare recipient device via the communication network. As used herein, “configuration parameters” refer to parameters that customize the healthcare provider's recommendations based on the healthcare recipient's inputs on the healthcare recipient device. The configuration parameters comprise, for example, changes made to the personalized prescription by the healthcare recipient to customize the recommendations of the healthcare provider.

In various embodiments, at block 104, the HAMS may monitor adherence of the healthcare recipient to the dynamically updated personalized prescription in communication with the healthcare mobile application on the healthcare recipient device via the communication network. By monitoring adherence of the healthcare recipient to the dynamically updated personalized prescription, the HAMS may monitor the adherence of the healthcare recipient to the healthcare plan.

The HAMS dynamically generates an adherence report based on the monitored adherence of the healthcare recipient to the dynamically updated personalized prescription. Furthermore, the HAMS transmits the dynamically generated adherence report to the healthcare provider device via the communication network for a review of the adherence of the healthcare recipient to the healthcare plan by the healthcare provider. In an embodiment, the HAMS integrates the dynamically generated adherence report with medical records of the healthcare recipient in a database for allowing a healthcare provider to determine a health condition of the healthcare recipient and predict healthcare requirements. The integration of the generated adherence report with the healthcare recipient's medical records in a database provides an insight into the healthcare recipient's life and behavior, which can be used by the healthcare provider to provide customized interventions to change or improve the healthcare recipient's behavior. Predicting the healthcare requirements comprises predicting steps to be taken by the healthcare recipient for a healthier lifestyle, predicting the likelihood of onset of diseases, predicting other health outcomes, etc. The HAMS allows healthcare providers to provide recommendations to healthcare recipients in a mobile application (app) format. Instead of a printed to-do list that a healthcare recipient must act on, the HAMS generates a mobile healthcare support environment that integrates the healthcare provider's recommendations into the healthcare recipient's schedule. The HAMS also provides a patient adherence platform for monitoring the adherence of a patient to a prescription provided by a physician.

In an example, a healthcare provider accesses the healthcare adherence monitoring system (HAMS) via a graphical user interface of the healthcare provider device and provides a personalized prescription for exercise, nutrition, and medication. In another example, the healthcare provider accesses the HAMS and drags and drops personalized components for exercise, nutrition, and medication in a mobile application format. The HAMS then transmits the personalized prescription electronically to the healthcare recipient, for example, a patient via the healthcare mobile application created by the HAMS. The patient accesses the personalized prescription via the healthcare mobile application and further customizes the recommendations. The HAMS tracks the patient's compliance and adherence to exercise, nutrition, and mediation as defined by the personalized prescription. In an embodiment, the HAMS provides a summary report to the patient on a continuous basis to keep him/her abreast of his/her compliance with the healthcare provider's recommendations. In another embodiment, the HAMS sends the adherence report, for example, in the form of a patient compliance report to the healthcare provider based on the tracked compliance and adherence of the patient to the personalized prescription. In another embodiment, the HAMS may store information on the tracked compliance and adherence of the patient to the exercise, nutrition, and medication related personalized prescription in an adherence data warehouse to provide more insight into the patient's life and behavior. The HAMS may also integrate information on the tracked compliance and adherence of the patient with medical records stored, for example, in electronic health records, electronic medical records, etc., of the patient to predict outcomes of the patient's behavior related to health.

According to various aspects, as mentioned above, the HAMS may store information on the tracked compliance and adherence of the patient to the personalized prescription in an adherence data warehouse. For example, in various embodiments, patient-generated adherence data may be stored in blocks or an adherence blockchain, thereby enabling personal and patient information into the hands of an individual. More particularly, the adherence blockchain may generally comprise a distributed database used to maintain a continuously growing list of records or blocks that include the patient-generated adherence data. Each block in the adherence blockchain may contain a timestamp and a link to a previous block, wherein various entities participating in the HAMS may adhere to a protocol to validate new blocks that are added to the adherence blockchain. For example, in various embodiments, the adherence blockchain may include a cryptographic hash of each record or block to prevent tampering and ensure data integrity. Furthermore, all stakeholders, including healthcare providers, healthcare recipients, etc. can view or add a new record associated with a particular patient, and patients may authorize sharing of records between and among stakeholders. As such, the adherence blockchain may essentially function as a ledger that can be used to record and verify patient-generated adherence data in a generally similar as blockchains used for the digital currency bitcoin. For example, in various embodiments, the adherence data may include information related to medication, exercise, nutrition, sleep patterns, heart rate, glucose, and/or other suitable data that can be polled from healthcare recipient devices and stored using the adherence blockchain. The adherence data stored as the blocks in the blockchain can then be converted to healthcoins or another suitable abstraction, which doctors, insurance agencies, and/or other suitable entities can use to regulate premiums, manage incentives, etc.

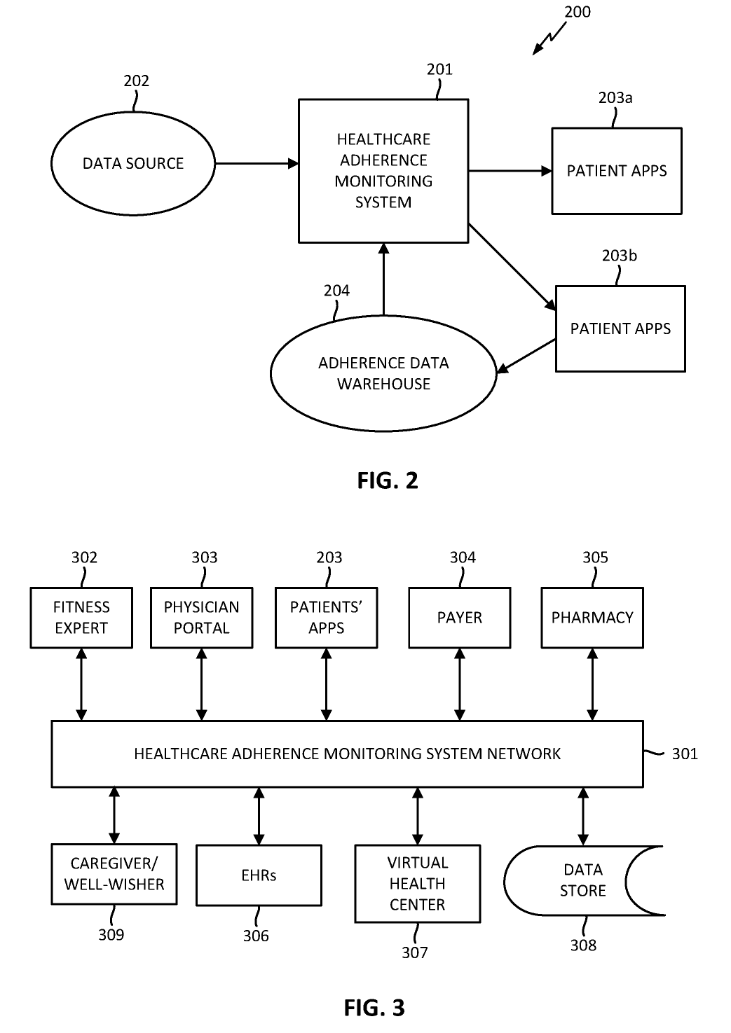

According to various aspects, FIG. 2 illustrates exemplary communication between the healthcare adherence monitoring system (HAMS) 201, a data source 202, for example, electronic health records, electronic medical records, etc., patient applications (apps) 203a and 203b, and an adherence data warehouse 204. A system 200 for monitoring adherence of a healthcare recipient, for example, a patient to a healthcare plan comprises the HAMS 201, healthcare mobile applications herein referred to as the “patient applications (apps)” 203a and 203b, and the adherence data warehouse 204 in operable communication with each other via a communication network (not shown). The HAMS 201 receives healthcare recommendations and personalized prescriptions based on information stored in the data source 202, for example, electronic health records, electronic medical records, etc., from a healthcare provider device, for example, a physician's smartphone. In an embodiment, the HAMS 201 creates patient applications 203a and 203b based on the received recommendations and prescriptions for use by patients on healthcare recipient devices as described in further detail above with respect to FIG. 1. The patient applications 203a and 203b on the healthcare recipient devices monitor the adherence of the patients to the recommendations and the prescriptions and generate adherence reports for the patients. The patient applications 203a and 203b send the adherence reports to the adherence data warehouse 204 for storing information on adherence of the patients to the recommendations and the prescriptions along with medical records of the patients. The adherence data warehouse 204 stores adherence or compliance data received from mobile and wearable devices (e.g., in an adherence blockchain, as described in further detail above). The HAMS 201 receives the adherence reports for the patients from the adherence data warehouse 204 and transmits the adherence reports to the healthcare provider device for allowing the healthcare provider to determine health conditions of the patients and predict healthcare requirements for the patients as disclosed in the detailed description of FIG. 1.

FIG. 3 illustrates exemplary healthcare entities connected to a communication network of the healthcare adherence monitoring system (HAMS) 201 exemplarily illustrated in FIG. 2, herein referred to as a “HAMS network”. The HAMS network 301 allows communication between multiple healthcare entities. The healthcare entities may comprise, without limitation, a fitness expert 302, a physician portal 303 for accessing medical records and adherence reports of patients, patients' applications 203, a payer 304 who pays for healthcare of a patient, and a pharmacy 305 that provides medications prescribed to the patients. The healthcare entities connected by the HAMS network 301 may further comprise electronic health records (EHRs) 306 of the patients, a virtual health center 307, and a data store 308 for storing the adherence reports of the patients (e.g., in an adherence blockchain, as described in further detail above).

Furthermore, as shown in FIG. 3, the healthcare entities connected by the HAMS network 301 may further comprise caregivers or well-wishers 309 of the patients. For example, in various embodiments, a patient may download a patient application 203 and enter medication, exercise, nutrition, and/or other suitable healthcare data as-prescribed or over-the-counter (OTC). The patient may optionally customize the recommendations as set forth in the patient application 203 and invite the one or more caregivers or well-wishers 309 to track his/her adherence to the recommendations. Accordingly, the caregiver(s) or well-wisher(s) 309 who receive the invitation from the patient may download the application 203 and view the information as entered by the patient. Alternatively and/or additionally, the caregiver(s)/well-wisher(s) 309 may enter medication, exercise, nutrition, and/or other suitable information as-prescribed on behalf of the patient and/or further customize the recommendations prescribed to the patient. The HAMS 201 may track the patient's compliance and adherence to the medication, exercise, nutrition, etc. set forth in the personalized prescription and provide reminders to the patients as well as the caregiver(s)/well-wisher(s) 309.

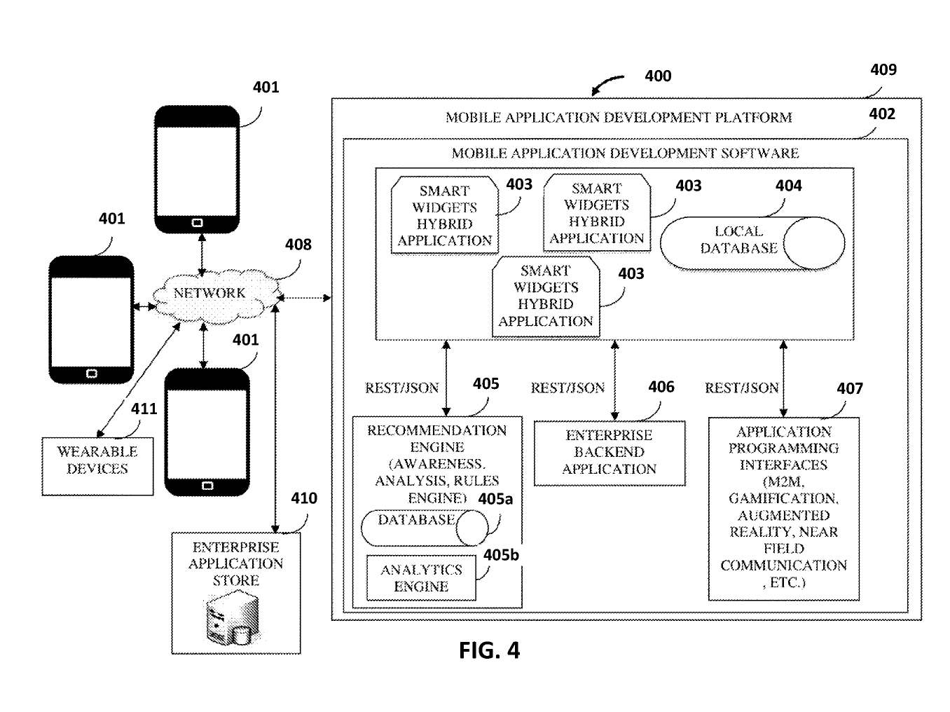

According to various aspects, FIG. 4 illustrates an exemplary high-level architecture diagram of a computer implemented system 400 for creating a mobile application for a user device 401, such as a healthcare mobile application accessible by a healthcare recipient device via a communication network for use in a healthcare adherence monitoring system (HAMS) as described above with respect to FIG. 1-3. The computer implemented system 400 disclosed herein comprises mobile application development software 402 accessible by multiple user devices 401, for example, personal computers, tablet computing devices, mobile computers, mobile phones, smart phones, etc., via a network 408, for example, the internet, a mobile communication network, etc. The mobile application development software 402 is also accessible by wearable devices 411. In an embodiment, the mobile application development software 402 is hosted on a mobile application development platform 409 accessible by multiple user devices 401 via the network 408. The mobile application development software 402 hosts multiple pre-coded software components, for example, smart widgets hybrid applications 403 also referred to as “smart widgets”, executable by at least one processor and insertable into the launched mobile application creation interface. The smart widgets 403 are adaptable based on context and behavioral elements. The smart widgets 403 are representations of a set of pre-written codes that perform predefined functions, for example, sales functions. The smart widgets 403 are, for example, programs coded in JavaScript®, which are prepackaged with client JavaScript® libraries by using advanced features of model view controller (MVC) design patterns. A model view controller is a computer user interface that separates a representation of information from the user's interaction with the information. The mobile application development software 402 implements reactive programming for adapting the smart widgets 403 and application programming interfaces (APIs) 407.

The mobile application development software 402 further comprises a recommendation engine 405, an enterprise backend application 406, application programming interfaces 407, and a local database 404. The local database 404 stores the smart widgets 403 and their metadata for the mobile application being created by power users. The mobile application development software 402 invokes the recommendation engine 405, the enterprise backend application 406, and the application programming interfaces 407, for example, via a JavaScript Object Notation (JSON) based representational state transfer (REST) architecture. The recommendation engine 405 generates one or more recommendations for addition of one or more characteristic objects associated with the mobile application based on a real time analysis and dynamic learning of selective data of similar mobile applications developed, for example, based on functionality, an industry, and/or a category related to the mobile application. The recommendation engine 405 comprises an analytics engine 405b that performs the real time analysis for addition of one or more characteristic objects associated with the mobile application. The mobile application development software 402 provides support for real time analysis and dynamic learning of selective data of similar mobile applications using in-memory databases, for example, 405a. The recommendation engine 405 further comprises a database 405a to store the generated recommendations. The enterprise backend application 406 adaptively configures the application programming interfaces 407 for a backend integration of the mobile application with the user device 401 for launching the mobile application on the user device 401. The mobile application development software 402 implements application programming interfaces 407 used for machine to machine (M2M) convergence, gamification, augmented reality, near field communication, etc., for backend integration of the mobile application with the user device 401. The mobile application development software 402 utilizes game design techniques, game thinking, and game mechanics to enhance non-game contexts in mobile application development. Augmented reality (AR) is a live, direct or indirect, view of a physical, real world environment whose elements are augmented by computer-generated sensory input such as sound, video, graphics, etc. The mobile application development software 402 transmits the created mobile applications for publishing and distribution, for example, to an enterprise application store 410. End users can download the created mobile applications by accessing the enterprise application store 410 directly from their user devices 401.

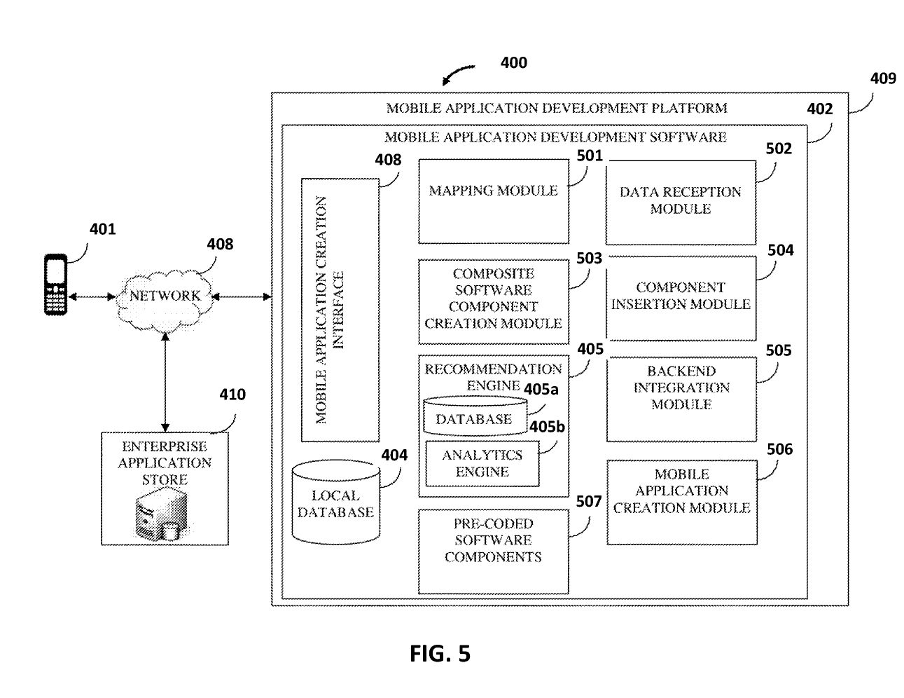

According to various aspects, FIG. 5 illustrates an exemplary computer implemented system 400 for creating a mobile application for a user device 401, such as a healthcare mobile application accessible by a healthcare recipient device via a communication network for use in a healthcare adherence monitoring system (HAMS) as described above with respect to FIG. 1-3. In particular, FIG. 5 illustrates various different modules, for example, 405, 405b, 501, 502, 503, 504, 505, 506, and 507 of the mobile application development software 402, which may be hosted, for example, on a mobile application development platform 409. The computer implemented system 400 disclosed herein comprises at least one processor, a non-transitory computer readable storage medium communicatively coupled to the processor, and the mobile application development software 402 executable by the processor. The mobile application development software 402 is accessible by a user device 401, for example, a mobile phone, a laptop, a tablet computing device, a wearable device 411 such as Google Glass® of Google Inc., the Apple Watch® of Apple Inc., etc., exemplarily illustrated in FIG. 4, via a network 408, for example, the internet, a mobile communication network, etc. The non-transitory computer readable storage medium is configured, for example, in the form of a database, for example, 404 to store the modules, for example, 501, 502, 503, 504, 505, 506, 507, etc., of the mobile application development software 402.

The mobile application development software 402 comprises pre-coded software components 507, a mapping module 501, a data reception module 502, a composite software component creation module 503, a component insertion module 504, a recommendation engine 405, a backend integration module 505, a mobile application creation module 506, and an analytics engine 405b. In an embodiment, the analytics engine 405b is incorporated in the recommendation engine 405. The mobile application development software 402 further comprises a mobile application creation interface 508 and a local database 404. The local database 404 stores the pre-coded software components 507. The pre-coded software components 507 are encapsulated in the mobile application creation interface 508 and are adaptable based on context and behavioral elements. The mapping module 501 dynamically maps the data to be rendered in the mobile application with one or more data sources. In an embodiment, the mapping module 501 dynamically populates the data sources in the mobile application creation interface 508 and maps one or more of the data sources to one or more of the pre-coded software components 507 for updating the pre-coded software components 507 and/or for creating one or more distinct software components based on inputs received from the user device 401. The data reception module 502 receives a selection of one of the preconfigured user interfaces or one of the predefined user interfaces from the user device 401 via the network 408 for launching the mobile application creation interface 508. The data reception module 502 further receives an indication of each of one or more of the pre-coded software components 507 selected by a user via the network 408.

The composite software component creation module 503 creates one or more composite software components by combining more than one of distinct software components selected from multiple component sources and/or the pre-coded software components 507. The component insertion module 504 inserts the selected pre-coded software components 507 and the created composite software components into the launched mobile application creation interface 508. The recommendation engine 405 generates one or more recommendations for addition of one or more characteristic objects associated with the mobile application based on a real time analysis and dynamic learning of selective data of similar mobile applications developed, for example, based on functionality, an industry, and/or a category related to the mobile application. The recommendation engine 405 comprises a database 405a for storing the generated recommendations. The recommendation engine 405 further dynamically updates the generated recommendations for addition of the characteristic objects associated with the mobile application based on a selection of one or more supplementary characteristic objects received from the user device 401. The backend integration module 505 adaptively configures one or more application programming interfaces 407 for a backend integration of the mobile application with the user device 401 for operating the mobile application on the user device 401. The backend integration module 505 further connects the created mobile application to multiple backend databases via the network 408 for creating enhanced mobile applications.

The mobile application creation module 506 creates the mobile application in the launched mobile application creation interface 508 using one or more of the inserted pre-coded software components 507 and the created composite software components, the generated recommendations, the dynamically mapped data, and the adaptively configured application programming interfaces 407. The mobile application creation module 506 further displays real time data simulations of the mobile application in the launched mobile application creation interface 508 during each stage of creation of the mobile application. In an embodiment, the mobile application creation module 506 transmits the created mobile application to another user specified by the user who created the mobile application for approval. On receiving approval, the mobile application creation module 506 transmits the approved mobile application to an enterprise application store 410 for publishing on the enterprise application store 410. The analytics engine 405b collects real time usage data for tracking and categorizing users of the created mobile application and for providing real time analytics, for example, to target surveys, advertisements, etc.

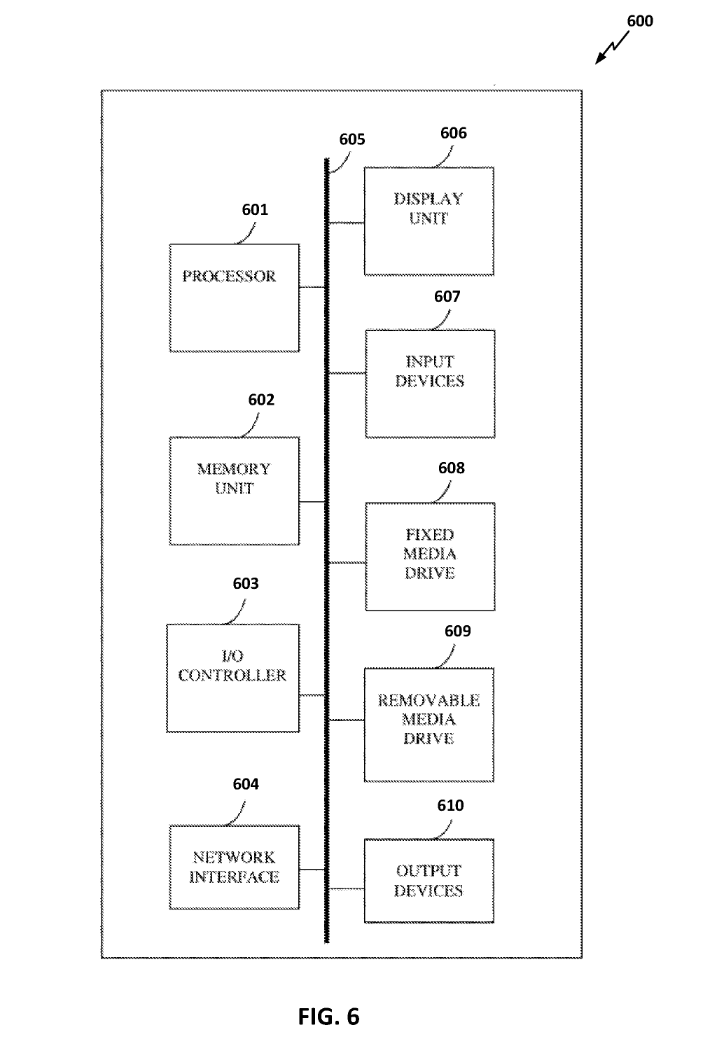

According to various aspects, FIG. 6 illustrates an exemplary computer system 600 employed by the mobile application development software for creating a mobile application for a user device, such as a healthcare mobile application accessible by a healthcare recipient device via a communication network for use in a healthcare adherence monitoring system (HAMS) as described above with respect to FIG. 1-3.

The computer system 600 comprises, for example, a processor 601, a memory unit 602 for storing programs and data, an input/output (I/O) controller 603, a network interface 604, a data bus 605, a display unit 606, input devices 607, a fixed media drive 608, a removable media drive 609 for receiving removable media, output devices 610, etc. The term “processor” refers to any one or more microprocessors, central processing unit (CPU) devices, finite state machines, computers, microcontrollers, digital signal processors, logic, a logic device, an electronic circuit, an application specific integrated circuit (ASIC), a field-programmable gate array (FPGA), a chip, etc., or any combination thereof, capable of executing computer programs or a series of commands, instructions, or state transitions. The processor 601 may also be implemented as a processor set comprising, for example, a general purpose microprocessor and a math or graphics co-processor. The computer implemented system 400 disclosed herein is not limited to the computer system 600 employing the processor 601. The computer system 600 may also employ a controller or a microcontroller.

The memory unit 602 is used for storing programs, applications, and data. For example, the mapping module 501, the data reception module 502, the composite software component creation module 503, the component insertion module 504, the recommendation engine 405, the backend integration module 505, the mobile application creation module 506, the pre-coded software components 507, and the analytics engine 405b, etc., of the mobile application development software 402 are stored in the memory unit 602 of the computer system 600 of the mobile application development software 402. The memory unit 602 is, for example, a random access memory (RAM) or another type of dynamic storage device that stores information and instructions for execution by the processor 601. The memory unit 602 also stores temporary variables and other intermediate information used during execution of the instructions by the processor 601. The computer system 600 further comprises a read only memory (ROM) or another type of static storage device that stores static information and instructions for the processor 601.

The I/O controller 603 controls input actions and output actions performed by the mobile application development software 402. The network interface 604 enables connection of the computer system 600 to the network 408. For example, the computer system 600 employed by the mobile application development software 402 connects to the network 408 via the network interface 604. In an embodiment, the network interface 604 is provided as an interface card also referred to as a line card. The network interface 604 comprises, for example, one or more of an infrared (IR) interface, a wireless local area network (WLAN) interface, a universal serial bus (USB) interface, a FireWire® interface, an Ethernet interface, a frame relay interface, a cable interface, a digital subscriber line (DSL) interface, a token ring interface, a peripheral controller interconnect (PCI) interface, a local area network (LAN) interface, a wide area network (WAN) interface, interfaces using serial protocols, interfaces using parallel protocols, and Ethernet communication interfaces, asynchronous transfer mode (ATM) interfaces, a high-speed serial interface (HSSI), a fiber distributed data interface (FDDI), interfaces based on transmission control protocol (TCP)/internet protocol (IP), interfaces based on wireless communications technology such as satellite technology, radio frequency (RF) technology, near field communication, etc. The data bus 605 permits communications between the modules, for example, 501, 502, 503, 504, 505, 506, 507, 405, 405b, etc., of the mobile application development software 402.

The display unit 606 displays information, display interfaces, user interface elements such as data fields, text boxes, windows, icons representing the pre-coded software components 507, etc. The display unit 606 comprises, for example, a liquid crystal display, a plasma display, an organic light emitting diode (OLED) based display, etc. The input devices 607 are used for inputting data into the computer system 600. For example, a user may enter a selection of one of the preconfigured user interfaces or the predefined user interfaces using the input devices 607. The input devices 607 are, for example, a keyboard such as an alphanumeric keyboard, a microphone, a joystick, a pointing device such as a computer mouse, a touch pad, a light pen, a physical button, a pointing device, a touch sensitive display device, a track ball, a pointing stick, any device capable of sensing a tactile input, etc.

Computer applications and programs are used for operating the computer system 600. The programs are loaded onto the fixed media drive 608 and into the memory unit 602 of the computer system 600 via the removable media drive 609. In an embodiment, the computer applications and programs may be loaded directly via the network 408. Computer applications and programs are executed by double clicking a related icon displayed on the display unit 606 using one of the input devices 607. The output devices 610 output the results of operations performed by the mobile application development software 402. For example, the mobile application development software 402 displays real time data simulations of the mobile application in the launched mobile application creation interface 508 during each stage of creation of the mobile application using the output devices 610.

The processor 601 executes an operating system for performing multiple tasks. The operating system is responsible for management and coordination of activities and sharing of resources of the computer system 600. The operating system further manages security of the computer system 600, peripheral devices connected to the computer system 600, and network connections. The operating system employed on the computer system 600 recognizes, for example, inputs provided by the users using one of the input devices 607, the output display, files, and directories stored locally on the fixed media drive 608, for example, a hard drive. The operating system on the computer system 600 executes different programs using the processor 601. The processor 601 and the operating system together define a computer platform for which application programs in high level programming languages are written.

The processor 601 retrieves instructions for executing the modules, for example, 501, 502, 503, 504, 505, 506, 507, 405, 405b, etc., of the mobile application development software 402 from the memory unit 602. A program counter determines the location of the instructions in the memory unit 602. The program counter stores a number that identifies the current position in the program of each of the modules, for example, 501, 502, 503, 504, 505, 506, 507, 405, 405b, etc., of the mobile application development software 402. The instructions fetched by the processor 601 from the memory unit 602 after being processed are decoded. The instructions are stored in an instruction register in the processor 601. After processing and decoding, the processor 601 executes the instructions. For example, the mapping module 501 defines instructions for dynamically mapping the data to be rendered in the mobile application with one or more data sources. The mapping module 501 also defines instructions for dynamically populating the data sources in the mobile application creation interface 508 and mapping one or more of the data sources to one or more of the pre-coded software components 507 for updating the pre-coded software components 507 and/or creating distinct software components based on inputs received from the user device 401. The data reception module 502 defines instructions for receiving a selection of one of the preconfigured user interfaces or the predefined user interfaces from the user device 401 via the network 408 for launching the mobile application creation interface 508. Furthermore, the data reception module 502 defines instructions for receiving an indication of each of the selected pre-coded software components 507 from the user device 401 via the network 408. The composite software component creation module 503 defines instructions for creating one or more composite software components by combining more than one of distinct software components selected from multiple component sources and/or the pre-coded software components 507.

The component insertion module 504 defines instructions for inserting one or more of the selected pre-coded software components 507 and the created composite software components into the launched mobile application creation interface 508. The recommendation engine 405 defines instructions for generating one or more recommendations for addition of one or more characteristic objects associated with the mobile application based on a real time analysis and dynamic learning of selective data of similar mobile applications developed, for example, based on functionality, an industry, and/or a category related to the mobile application. Furthermore, the recommendation engine 405 defines instructions for dynamically updating the generated recommendations for addition of the characteristic objects associated with the mobile application based on a selection of one or more supplementary characteristic objects received from the user device 401.

The backend integration module 505 defines instructions for adaptively configuring one or more application programming interfaces 407 for a backend integration of the mobile application with the user device 401 for launching the mobile application on the user device 401. The mobile application creation module 506 defines instructions for creating the mobile application in the launched mobile application creation interface 508 using one or more of the inserted pre-coded software components 507 and the created composite software components, the generated recommendations, the dynamically mapped data, and the adaptively configured application programming interfaces 407. Furthermore, the mobile application creation module 506 defines instructions for displaying real time data simulations of the mobile application in the launched mobile application creation interface 508 during each stage of creation of the mobile application. The backend integration module 505 defines instructions for connecting the created mobile application to multiple backend databases via the network 408 for creating enhanced mobile applications. The analytics engine 405b defines instructions for collecting real time usage data for tracking and categorizing users of the created mobile application and providing real time analytics, for example, to target surveys, advertisements, etc.

The processor 601 of the computer system 600 employed by the mobile application development software 402 retrieves the instructions defined by the mapping module 501, the data reception module 502, the composite software component creation module 503, the component insertion module 504, the recommendation engine 405, the backend integration module 505, the mobile application creation module 506, the analytics engine 405b, etc., of the mobile application development software 402, and executes the instructions, thereby performing one or more processes defined by those instructions.

At the time of execution, the instructions stored in the instruction register are examined to determine the operations to be performed. The processor 601 then performs the specified operations. The operations comprise arithmetic operations and logic operations. The operating system performs multiple routines for performing a number of tasks required to assign the input devices 607, the output devices 610, and memory for execution of the modules, for example, 501, 502, 503, 504, 505, 506, 507, 509, 405, 405b, etc., of the mobile application development software 402. The tasks performed by the operating system comprise, for example, assigning memory to the modules, for example, 501, 502, 503, 504, 505, 506, 507, 509, 405, 405b, etc., of the mobile application development software 402, and to data used by the mobile application development software 402, moving data between the memory unit 602 and disk units, and handling input/output operations. The operating system performs the tasks on request by the operations and after performing the tasks, the operating system transfers the execution control back to the processor 601. The processor 601 continues the execution to obtain one or more outputs. The outputs of the execution of the modules, for example, 501, 502, 503, 504, 505, 506, 507, 509, 405, 405b, etc., of the mobile application development software 402 are displayed to the user on the display unit 606.

For purposes of illustration, the detailed description refers to the mobile application development software 402 being run locally on the computer system 600; however the scope of the computer implemented method and system 400 disclosed herein is not limited to the mobile application development software 402 being run locally on the computer system 600 via the operating system and the processor 601, but may be extended to run remotely over the network 408 by employing a web browser and a remote server, a mobile phone, or other electronic devices. One or more portions of the computer system 600 may be distributed across one or more computer systems (not shown) coupled to the network 408.

Those skilled in the art will appreciate that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Further, those skilled in the art will appreciate that the various illustrative logical blocks, modules, circuits, and algorithm steps described in connection with the aspects disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted to depart from the scope of the various aspects and embodiments described herein.

The various illustrative logical blocks, modules, and circuits described in connection with the aspects disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices (e.g., a combination of a DSP and a microprocessor, a plurality of microprocessors, one or more microprocessors in conjunction with a DSP core, or any other such configuration).

The methods, sequences, and/or algorithms described in connection with the aspects disclosed herein may be embodied directly in hardware, in a software module executed by a processor, or in a combination of the two. A software module may reside in RAM, flash memory, ROM, EPROM, EEPROM, registers, hard disk, a removable disk, a CD-ROM, or any other form of non-transitory computer-readable medium known in the art. An exemplary non-transitory computer-readable medium may be coupled to the processor such that the processor can read information from, and write information to, the non-transitory computer-readable medium. In the alternative, the non-transitory computer-readable medium may be integral to the processor. The processor and the non-transitory computer-readable medium may reside in an ASIC. The ASIC may reside in an IoT device. In the alternative, the processor and the non-transitory computer-readable medium may be discrete components in a user terminal.

In one or more exemplary aspects, the functions described herein may be implemented in hardware, software, firmware, or any combination thereof. If implemented in software, the functions may be stored on or transmitted over as one or more instructions or code on a non-transitory computer-readable medium. Computer-readable media may include storage media and/or communication media including any non-transitory medium that may facilitate transferring a computer program from one place to another. A storage media may be any available media that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer. Also, any connection is properly termed a computer-readable medium. For example, if the software is transmitted from a website, server, or other remote source using a coaxial cable, fiber optic cable, twisted pair, DSL, or wireless technologies such as infrared, radio, and microwave, then the coaxial cable, fiber optic cable, twisted pair, DSL, or wireless technologies such as infrared, radio, and microwave are included in the definition of a medium. The term disk and disc, which may be used interchangeably herein, includes CD, laser disc, optical disc, DVD, floppy disk, and Blu-ray discs, which usually reproduce data magnetically and/or optically with lasers. Combinations of the above should also be included within the scope of computer-readable media.

While the foregoing disclosure shows illustrative aspects and embodiments, those skilled in the art will appreciate that various changes and modifications could be made herein without departing from the scope of the disclosure as defined by the appended claims. Furthermore, in accordance with the various illustrative aspects and embodiments described herein, those skilled in the art will appreciate that the functions, steps, and/or actions in any methods described above and/or recited in any method claims appended hereto need not be performed in any particular order. Further still, to the extent that any elements are described above or recited in the appended claims in a singular form, those skilled in the art will appreciate that singular form(s) contemplate the plural as well unless limitation to the singular form(s) is explicitly stated.

What is claimed is:

1. A method for monitoring adherence of a healthcare recipient to a healthcare plan, comprising:
receiving a personalized prescription at a healthcare adherence monitoring system, wherein the personalized prescription received at the healthcare adherence monitoring system comprises one or more medication, exercise, and nutrition recommendations received from a healthcare provider device via a communication network;
incorporating, at the healthcare adherence monitoring system, the personalized prescription into a healthcare mobile application;
sending, by the healthcare adherence monitoring system, the healthcare mobile application incorporating the personalized prescription to a healthcare recipient device via the communication network; and
monitoring, at the healthcare adherence monitoring system, adherence of the healthcare recipient to the personalized prescription based on adherence data received from the healthcare mobile application on the healthcare recipient device via the communication network.

2. The method recited in claim 1, further comprising:
providing, by the healthcare adherence monitoring system, an interface that enables the healthcare provider device to drag and drop one or more pre-coded prescription components to create the healthcare mobile application, wherein the pre-coded prescription components include executable computer program code encapsulated to perform predefined functions corresponding to the personalized prescription.

3. The method recited in claim 1, further comprising:
dynamically updating the personalized prescription based on one or more configuration parameters received from the healthcare mobile application on the healthcare recipient device via the communication network;
dynamically generating an adherence report based on the monitored adherence of the healthcare recipient to the dynamically updated personalized prescription; and
transmitting the adherence report to the healthcare recipient device via the communication network.

4. The method recited in claim 1, further comprising:
dynamically updating the personalized prescription based on one or more configuration parameters received from the healthcare mobile application on the healthcare recipient device via the communication network;
dynamically generating an adherence report based on the monitored adherence of the healthcare recipient to the dynamically updated personalized prescription; and
transmitting the adherence report to the healthcare provider device via the communication network.

5. The method recited in claim 1, further comprising:
storing, at the healthcare adherence monitoring system, data related to the adherence of the healthcare recipient to the personalized prescription, wherein the data related to the adherence of the healthcare recipient to the personalized prescription is accessible to the healthcare provider device via the communication network;
receiving, at the healthcare adherence monitoring system, one or more customized interventions from the healthcare provider device via the communication network, the one or more customized interventions including one or more steps to be taken by the healthcare recipient for a healthier lifestyle; and
sending the one or more customized interventions to the healthcare recipient device via the communication network.

6. The method recited in claim 1, further comprising:
storing, at the healthcare adherence monitoring system, data related to the adherence of the healthcare recipient to the personalized prescription, wherein the stored data related to the adherence of the healthcare recipient to the personalized prescription is integrated with one or more medical records of the healthcare recipient; and
predicting, at the healthcare adherence monitoring system, a likelihood of one or more health outcomes for the healthcare recipient based on the stored data related to the adherence of the healthcare recipient to the personalized prescription in combination with the one or more medical records of the healthcare recipient.

7. The method recited in claim 1, further comprising:
receiving, at the healthcare adherence monitoring system, information from the healthcare recipient device inviting one or more caregivers to track the adherence of the healthcare recipient to the personalized prescription; and
sending, by the healthcare adherence monitoring system, the healthcare mobile application incorporating the personalized prescription to one or more devices associated with the one or more caregivers via the communication network.

8. The method recited in claim 7, further comprising:
monitoring, at the healthcare adherence monitoring system, adherence of the healthcare recipient to the personalized prescription based on adherence data received from the healthcare mobile application on the one or more devices associated with the one or more caregivers via the communication network.

9. The method recited in claim 1, further comprising:
storing, at the healthcare adherence monitoring system, the adherence data received from the healthcare mobile application on the healthcare recipient device as one or more blocks in an adherence blockchain, wherein each of the one or more blocks in the adherence blockchain includes a cryptographic hash of the adherence data stored in the respective block.

10. The method recited in claim 9, further comprising:
converting the adherence data stored as the one or more blocks in the adherence blockchain into healthcoins, wherein the healthcoins are accessible to one or more entities via the communication network to prescribe or regulate one or more of healthcare premiums or incentives for adhering to the personalized prescription.

11. A healthcare adherence monitoring system, comprising:
a network interface configured to communicate via a communication network; and
one or more processors configured to:
receive, via the network interface, a personalized prescription comprising one or more medication, exercise, and nutrition recommendations received from a healthcare provider device;
incorporate the personalized prescription into a healthcare mobile application;
send, via the network interface, the healthcare mobile application incorporating the personalized prescription to a healthcare recipient device; and
monitor adherence of the healthcare recipient to the personalized prescription based on adherence data received from the healthcare mobile application on the healthcare recipient device via the communication network.

12. The healthcare adherence monitoring system recited in claim 11, wherein the one or more processors are further configured to:
provide an interface that enables the healthcare provider device to drag and drop one or more pre-coded prescription components to create the healthcare mobile application, wherein the pre-coded prescription components include executable computer program code encapsulated to perform predefined functions corresponding to the personalized prescription.

13. The healthcare adherence monitoring system recited in claim 11, wherein the one or more processors are further configured to:
dynamically update the personalized prescription based on one or more configuration parameters received from the healthcare mobile application on the healthcare recipient device via the communication network;
dynamically generate an adherence report based on the monitored adherence of the healthcare recipient to the dynamically updated personalized prescription; and
transmit, via the network interface, the adherence report to one or more of the healthcare recipient device or the healthcare provider device via the communication network.

14. The healthcare adherence monitoring system recited in claim 11, further comprising:
an adherence data warehouse comprising at least one memory configured to store data related to the adherence of the healthcare recipient to the personalized prescription,
wherein the data related to the adherence of the healthcare recipient to the personalized prescription is accessible to the healthcare provider device via the communication network, and
wherein the one or more processors are further configured to:
receive, via the network interface, one or more customized interventions from the healthcare provider device, the one or more customized interventions including one or more steps to be taken by the healthcare recipient for a healthier lifestyle; and
send, via the network interface, the one or more customized interventions to the healthcare recipient device.

15. The healthcare adherence monitoring system recited in claim 11, further comprising:
an adherence data warehouse comprising at least one memory configured to store data related to the adherence of the healthcare recipient to the personalized prescription, wherein the stored data related to the adherence of the healthcare recipient to the personalized prescription is integrated with one or more medical records of the healthcare recipient; and
wherein the one or more processors are further configured to predict a likelihood of one or more health outcomes for the healthcare recipient based on the stored data related to the adherence of the healthcare recipient to the personalized prescription in combination with the one or more medical records of the healthcare recipient.

16. The healthcare adherence monitoring system recited in claim 11, wherein the one or more processors are further configured to:
receive, via the network interface, information from the healthcare recipient device inviting one or more caregivers to track the adherence of the healthcare recipient to the personalized prescription; and
send, via the network interface, the healthcare mobile application incorporating the personalized prescription to one or more devices associated with the one or more caregivers via the communication network.

17. The healthcare adherence monitoring system recited in claim 16, wherein the one or more processors are further configured to:
monitor adherence of the healthcare recipient to the personalized prescription based on adherence data received from the healthcare mobile application on the one or more devices associated with the one or more caregivers via the communication network.

18. The healthcare adherence monitoring system recited in claim 11, further comprising:
an adherence data warehouse comprising at least one memory configured to store the adherence data received from the healthcare mobile application on the healthcare recipient device as one or more blocks in an adherence blockchain, wherein each of the one or more blocks in the adherence blockchain includes a cryptographic hash of the adherence data stored in the respective block.

19. The healthcare adherence monitoring system recited in claim 18, wherein the one or more processors are further configured to:
convert the adherence data stored as the one or more blocks in the adherence blockchain into healthcoins, wherein the healthcoins are accessible to one or more entities via the communication network to prescribe or regulate one or more of healthcare premiums or incentives for adhering to the personalized prescription.

20. A non-transitory computer-readable storage medium, comprising computer-executable code configured to cause at least one processor to:
receive a personalized prescription, wherein the personalized prescription comprises one or more medication, exercise, and nutrition recommendations received from a healthcare provider device via a communication network;
incorporate the personalized prescription into a healthcare mobile application;
send the healthcare mobile application incorporating the personalized prescription to a healthcare recipient device via the communication network; and
monitor adherence of the healthcare recipient to the personalized prescription based on adherence data received from the healthcare mobile application on the healthcare recipient device via the communication network.
